(19) United States
(12) Patent Application Publication
(10) Pub. No.: US 2010/0049073 A1
(43) Pub. Date: Feb. 25, 2010

(54) Method, System, and Computer Program for Determining if a Subject is Snoring

(76) Inventor: Gaby Bader, Goteborg (SE)

Correspondence Address: HARNESS, DICKEY & PIERCE, P.L.C., P.O. BOX 8910, RESTON, VA, 20195, US

(21) Appl. No.: 11/922,987

(22) PCT Filed: Jun. 28, 2006
(86) PCT No.: PCT/SE2006/000789

§ 371 Date: Sep. 23, 2009

Related U.S. Application Data
(60) Provisional application No. 60/708,772, filed on Aug. 17, 2005.

(30) Foreign Application Priority Data
Jun. 30, 2005 (SE) 0501527-6

(51) Int. Cl. A61B 5/08

(52) U.S. Cl. 600/529

(57) ABSTRACT

In one embodiment of the present invention, a method for determining if a subject is snoring is disclosed, including the steps of: determining if the subject is present in a bed; determining if there is snoring; determining if snoring is emitted from the subject; and generating an output signal, wherein the output signal is dependent on if the snoring is emitted from the subject. A computer program arranged to cause a computer to perform the steps of the method is also disclosed. Further, a system for snoring determination is disclosed, comprising a processor, an audio input means providing an audio signal to the processor, and a sensor pad arranged in a bed, the sensor pad providing a sensor pad signal to the processor, wherein the processor comprises devices for determination of presence of a subject from the sensor pad signal; devices for determination of snoring from the audio signal; devices for determination if the subject is emitting the snoring from the sensor pad signal and the audio signal; and an output arranged to provide an output signal according to determinations of the devices for determination of presence, devices for determination of snoring, and devices for determination is the subject is emitting the snoring.

### TECHNICAL FIELD

The present invention relates to a method for determining if a subject is snoring, and a system and computer program for the same.

### BACKGROUND OF THE INVENTION

Beds for avoiding snoring is known from e.g. the Swedish patent No. 524903. Said patent discloses that a registering unit may be programmed to detect if a person snores, and a proper algorithm can then be used to detect disrupted breathing when the person is asleep by registering amount and duration. A control signal is caused by durable snoring, detected by sound recording, and activates elevation of a head end of the bed. However, there is a problem to accurately determine if the person is snoring. It is therefore a desire to improve detection of snoring.

### SUMMARY OF THE INVENTION

It is therefore an object of the present invention to provide improved detection of snoring.

The above object is achieved according to a first aspect of the present invention by a method for determining if a subject is snoring, comprising the steps of:

determining if said subject is present in a bed;

determining if there is snoring;

determining if snoring is emitted from said subject; and

generating an output signal, wherein said output signal is dependent on if said snoring is emitted from said subject.

An advantage of this is that it is clearly determined that the snoring is emitted from a sleeping person present in the bed.

In this context, the term “bed” should be construed as any place used by a person or animal for sleep or rest, i.e. the “bed” could as well be a bed, a sofa, a comfy armchair, etc.

The method may further comprise the step of determining if said subject is asleep, wherein said output signal is dependent on if said subject is asleep. The step of determining if the subject is asleep may comprise the steps of measuring physiological parameters of said subject, and processing the measured physiological parameters. The physiological parameters may be associated with breathing, heartbeat, or movements, or any combination thereof.

An advantage of this is that a higher degree of certainty that it is the subject that is snoring, since snoring is related to sleeping subjects.

The step of determining if a subject is present in said bed comprises the step of detecting a signal from a sensor pad of said bed.

An advantage of this is a high degree of certainty that a subject is present in the bed.

The step of determining if there is snoring may comprise the steps of: acquiring an audio signal in a vicinity of said bed; discriminating a physiological sound from said audio signal; and determining if said physiological sound comprises snoring sounds. The step of determining if said physiological sound comprises snoring sounds may comprise the steps of measuring physiological parameters of said subject, and processing measured physiological parameters. The physiological parameters may be associated with breathing, heartbeat, or movements, or any combination thereof.

An advantage of this is a high degree of certainty that snoring is present in or at least in the vicinity of the bed.

The step of determining if snoring is emitted from said subject may comprise the step of processing an audio signal from an audio input means arranged in a vicinity of said bed and a sensor pad signal from a sensor pad of said bed, wherein said processing comprises the step of determining if said audio signal and said sensor pad signal are synchrone. The processing may comprise the step of calculating a correlation between said audio signal and said sensor pad signal.

An advantage of this is that it enables determination that it is the subject that is snoring, and not any other person or animal being in the vicinity of the bed.

The above object is achieved according to a second aspect of the present invention by a system for snoring determination comprising a processor, an audio input means providing an audio signal to said processor, and a sensor pad arranged in a bed, said sensor pad providing a sensor pad signal to said processor,

wherein said processor comprises

means for determination of presence of a subject from said sensor pad signal;

means for determination of snoring from said audio signal;

means for determination if said subject is emitting said snoring from said sensor pad signal and said audio signal; and

an output arranged to provide an output signal according to determinations of said means for determination of presence, means for determination of snoring, and means for determination is said subject is emitting said snoring.

The processor may further comprise means for determination if said subject is asleep from said sensor pad signal, wherein said output also is dependent on said determination if said subject is asleep.

The audio input may be connected to a discriminator of said processor arranged to discriminate physiological sounds.

The sensor pad may be connected to a signal processor of said processor, said signal processor being arranged to extract physiological information.

The means for determination if said subject is emitting said snoring may comprise a correlator.

The above object is achieved according to a third aspect of the present invention by a computer program arranged to cause a computer to perform the steps of the method of the first aspect of the present invention, when downloaded and executed on said computer.

The advantages of the second and third aspect of the invention are essentially similar to those of the first aspect.

### BRIEF DESCRIPTION OF THE DRAWINGS

The above, as well as additional objects, features and advantages of the present invention, will be better understood through the following illustrative and non-limiting detailed description of preferred embodiments of the present invention, with reference to the appended drawings, wherein:

FIG. 1 illustrates an environment in which the present invention is to be performed;

FIG. 2 schematically illustrates functions of a system according to one embodiment of the present invention; and

FIG. 3 is a flow chart illustrating a method according to one embodiment of the present invention.

### DETAILED DESCRIPTION OF PREFERRED EMBODIMENTS

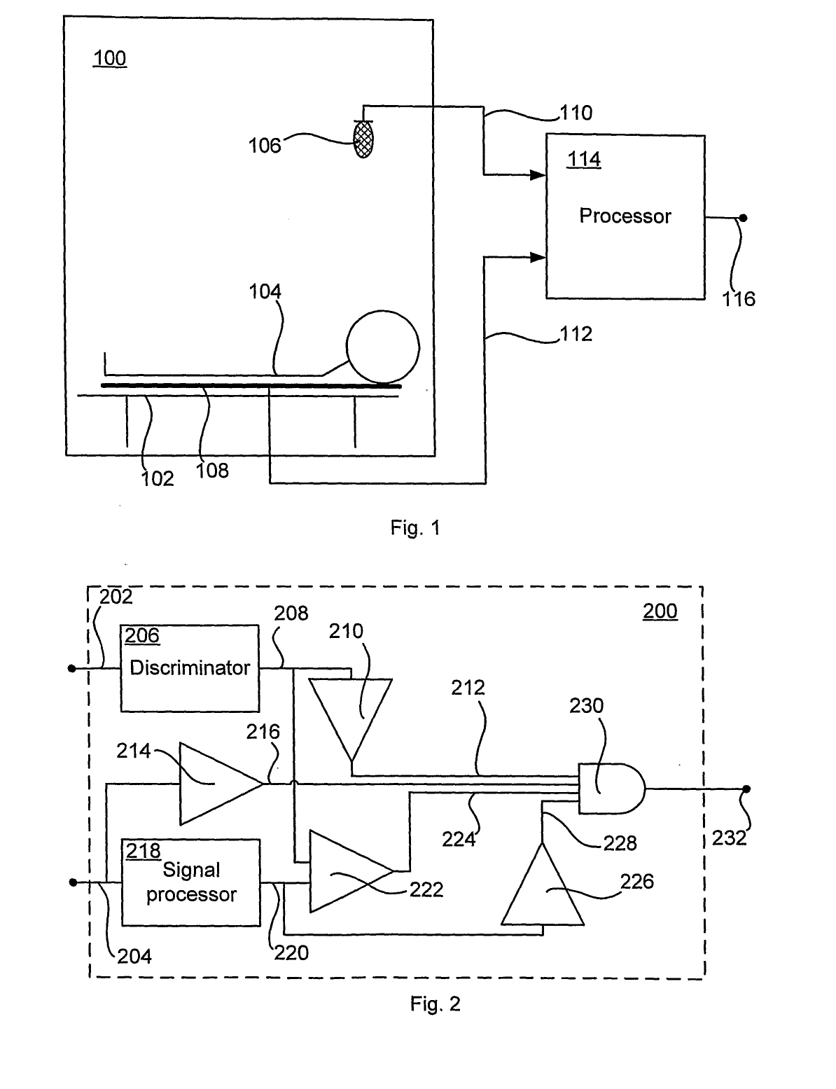

FIG. 1 illustrates an environment 100, e.g. a bedroom, which comprises a bed 102. In this context, the term “bed” should be construed as any place used by a person or animal for sleep or rest, i.e. the “bed” could as well be a bed, a sofa, a comfy armchair, etc. The bed 102 is arranged such that a subject 104, e.g. a person, can sleep in it.

In the environment 100 it is provided an audio registering unit 106, e.g. a microphone, to input sounds present in the vicinity of the bed 102. A sensor pad 108 is located in the bed 102 to sense mechanical occurrences, e.g. motions, pressure changes, accelerations, of the subject 104. The sensor pad 108 can comprise one or more piezo electric sensors, one or more capacitive sensors, or any other sensor types arranged to register mechanical occurences. When a plurality of sensors are used, they can be arranged as an array, or any other suitable configuration.

The audio registering unit 106 outputs an audio signal 110 and the sensor pad 108 outputs a sensor pad signal 112 to a processor 114, which is arranged to process the signals 110, 112 to determine if the subject actually is snoring. The processor provides an output signal 116, indicating if the sleeping subject in the bed is snoring.

FIG. 2 schematically illustrates functions of a processor 200 according to an embodiment of the present invention, e.g. the processor 112 of FIG. 1. The processor 200 receives an audio signal at an audio signal input 202, and receives a sensor pad input signal at a sensor pad signal input 204.

The audio signal at the audio signal input is provided to a discriminator 206 which is arranged to discriminate physiological sounds from the audio signal. A physiological sound signal 208 is output to an audio signal processing unit 210 which is arranged to determine if the physiological sounds comprise snoring. The audio signal processing unit 210 outputs a logical signal 212, which depends on determined snoring.

The sensor pad signal at the sensor pad signal input 204 is provided to a presence determination unit 214 which is arranged to determine if a subject is present in the bed, and output a logical signal 216 which depends on determined presence.

The sensor pad signal at the sensor pad signal input 204 is also provided to a sensor pad signal processing unit 218 which is arranged to process and extract physiological information from the sensor pad signal. The physiological information can be breathing, heartbeat, and/or movements. The sensor pad signal processing unit 218 outputs a complex signal 220 comprising extracted information.

A signal processing unit 222 receives the complex signal 220 and the physiological sound signal 208. The signal processing unit 222 is arranged to perform correlation between the complex signal 220 and the physiological sound signal 208. This correlation will discriminate physiological sounds related to the subject. Further, processing is performed in the signal processing unit 222 to determine snoring among the physiological sounds related to the subject, and a logical output signal 224 is provided, indicating if the subject snores.

The complex signal 220 is also provided to a processing unit 226 which is arranged to determine if the subject is asleep. The determination can be performed by analysing breathing, heartbeat, and/or movement information in the complex signal 220. The processing unit 226 outputs a logical signal 228 indicating if the subject is asleep or awake.

The logical signals 212, 216, 224, 228 are received by a determining unit 230 which outputs a logical output signal to an output 232 of the processor 200. Thus, the output 232 of the processor 200 indicates if the sleeping subject in the bed is snoring.

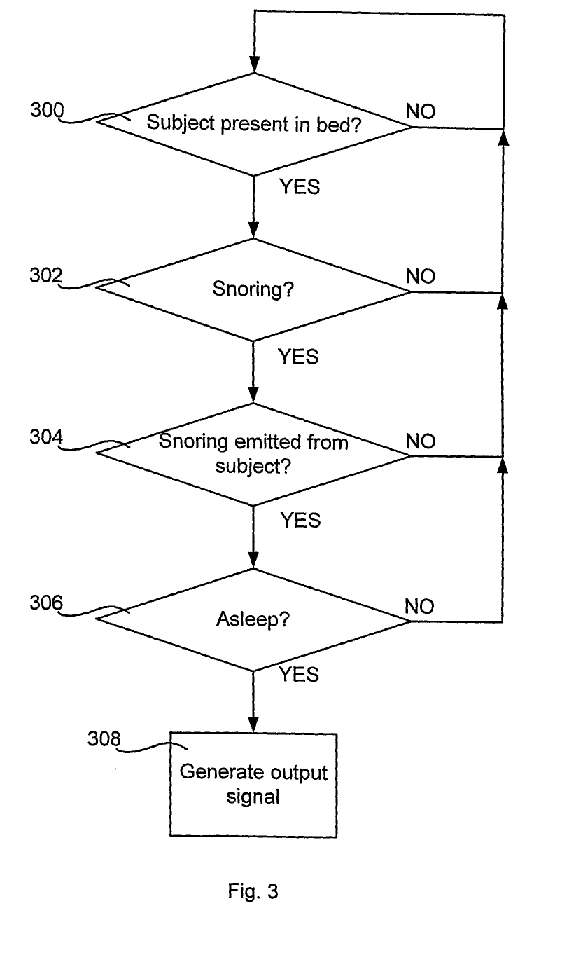

FIG. 3 is a flow chart illustrating a method according to one embodiment of the present invention. In a presence determination step 300 it is determined if a subject, e.g. a person, is present in a bed. In this context, the term “bed” should be construed as any place used by a person or animal for sleep or rest. It is determined in a snoring determination step 302 if there is any snoring in the vicinity of the bed. It should be noted that by this general approach, where all snoring in the vicinity of the bed is considered, the subject that is supposed to sleep is not affected. However, there could be others snoring in the vicinity of the bed, e.g. a room mate or a pet. Therefore, it is determined if the snoring is emitted from the subject in an idem determination step 304. To determine whether the subject is asleep or awake improves the degree of certainty that it is the subject that is snoring, since snoring is related to a sleeping subject, but hard to discriminate from other things, e.g. mumbling, coughing, hemming etc when the subject is awake. Therefore, it is determined in a sleeping determination step 306 if the subject is asleep. If these conditions are true, the method generates an output signal indicating that the sleeping subject in the bed is snoring in an output generation step 308. If any of these conditions is false, the method returns. It should be noted that these steps can be performed in any order or in parallel, as is the case for real-time systems. The only constraints is that there is available input data any step that is to be performed.

The method is well suited to be implemented as a computer program which, when downloaded and executed on a computer, performs the method of the invention together with the hardware of the above described system.

1. A method for determining if a subject is snoring, comprising the steps of:
determining if said subject is present in a bed;
determining if there is snoring;
determining if snoring is emitted from said subject, comprising the step of processing an audio signal from an audio input means arranged in a vicinity of said bed and a sensor pad signal from a sensor pad of said bed, wherein said processing comprises the step of determining if said audio signal and said sensor pad signal are synchrone;
determining if said subject is asleep, comprising measuring physiological parameters of said subject, and processing the measured physiological parameters; and
generating an output signal, wherein said output signal is dependent on if said snoring is emitted from said subject and if said subject is snoring.

2. The method according to claim 1, wherein said physiological parameters are associated with breathing, heartbeat, or movements, or any combination thereof.

3. The method according to claim 1, wherein said step of determining if a subject is present in said bed comprises the step of detecting a signal from a sensor pad of said bed.

4. The method according to claim 1, wherein said step of determining if there is snoring comprises the steps of:
acquiring an audio signal in a vicinity of said bed;
discriminating a physiological sound from said audio signal; and
determining if said physiological sound comprises snoring sounds.

5. The method according to claim 4, wherein said step of determining if said physiological sound comprises snoring sounds comprises the steps of
measuring physiological parameters of said subject; and
processing measured physiological parameters.

6. The method according to claim 5, wherein said physiological parameters are associated with breathing, heartbeat, or movements, or any combination thereof.

7. The method according to claim 1, wherein said processing comprises the step of calculating a correlation between said audio signal and said sensor pad signal.

8. A system for snoring determination comprising a processor, an audio input means providing an audio signal to said processor, and a sensor pad arranged in a bed, said sensor pad providing a sensor pad signal to said processor,
wherein said processor comprises
means for determination of presence of a subject from said sensor pad signal;
means for determination of snoring from said audio signal;
means for determination if said subject is emitting said snoring from said sensor pad signal and said audio signal, wherein said means for determination if said subject is emitting said snoring comprises a correlator; and
an output arranged to provide an output signal according to determinations of said means for determination of presence, means for determination of snoring, and means for determination if said subject is emitting said snoring.

9. The system according to claim 8, wherein said processor further comprises means for determination if said subject is asleep from said sensor pad signal, wherein said output signal also is dependent on said determination if said subject is asleep.

10. The system according to claim 8, wherein said audio input is connected to a discriminator of said processor arranged to discriminate physiological sounds.

11. The system according to claim 8, wherein said sensor pad is connected to a signal processor of said processor, said signal processor being arranged to extract physiological information.

12. A computer program arranged to cause a computer to perform the steps of claim 1, when downloaded and executed on said computer.

13. (canceled)

14. (canceled)

15. (canceled)

16. (canceled)
